(19) United States
(12) Patent Application Publication
(10) Pub. No.: US 2026/0325237 A1
(43) Pub. Date: Oct. 22, 2026

(54) Robot arm, ultrasonic robot and method for controlling a robot arm

(71) Applicant: DEUTSCHES ZENTRUM FÜR LUFT- UND RAUMFAHRT E.V., Bonn (DE)

(72) Inventors: Christopher Schlenk, Müchen (DE); Ribin Balachandran, Weßling (DE); Julian Klodmann, Müchen (DE)

(73) Assignee: Deutsches Zentrum Für Luft-Und Raumfahrt E.V., Bonn (DE)

(21) Appl. No.: 18/875,232

(22) PCT Filed: Nov. 16, 2023
(86) PCT No.: PCT/EP2023/082071

§ 371 Date: Dec. 16, 2024

(30) Foreign Application Priority Data
Nov. 17, 2022 (DE) 10 2022 130 514.7

(51) Int. Cl. A61B 34/32; A61B 8/00; A61B 17/34; A61B 34/30; A61B 90/00

(52) U.S. Cl. CPC A61B 34/32; A61B 8/4218; A61B 17/34; A61B 90/06; A61B 2034/306; A61B 2090/066

(57) ABSTRACT

A robot arm having a robot structure, the robot structure comprising an arm link chain having at least one arm link and at least one joint, and a tool connected to the arm link chain for connection to at least one end effector; at least one end effector sensor for detecting forces and/or torques acting on an end effector connected to the tool; and at least one structure sensor for detecting forces and/or torques acting on the robot structure. Furthermore, an ultrasonic robot and a method for controlling a robot arm.

### BACKGROUND

### 1. Field of the Disclosure

The disclosure relates to a robot arm, an ultrasonic robot and a method for controlling a robot arm.

### 2. Discussion of the Background Art

Robot arms are often used in industrial applications, such as automotive manufacturing, or in medical applications, such as robot-assisted surgery.

The target trajectories of a lightweight robot, e.g., a robot arm, can basically be generated in three ways:
Autonomy: The system autonomously calculates the desired trajectories and monitors compliance with them. This requires precise knowledge of the robot's environment, either because it is very structured and static, or because the robot can acquire all relevant information with the aid of appropriate sensors. Furthermore, the ability to react appropriately to unforeseen disturbances is generally assumed.
Teleoperation: The human user is remote from the robot arm and commands its movements by means of a suitable input device.
Hands-on control/physical human-robot interaction: The human user interacts directly with the robot arm to affect its movements and behavior.

In the medical field, with its unstructured environment and often direct contact between the robot system and patient, physical human-robot interaction offers advantages in numerous applications.

In contrast to systems with autonomy, there are the following advantages: As the sensory abilities and expertise of the human user are incorporated into the robot control, the number of sensor systems in the robotic system can be significantly reduced. As the human user can intervene at any time in the event of disturbances or unforeseen situations, the system's control software can be considerably simplified.

In contrast to systems with teleoperation, there are the following advantages: The human user can interact with the patient and the robot system at the same time. This gives the human user the best possible information about the surgical procedure at any time and full control over the further procedure, in particular up to the point where robotic support is dispensed with and the procedure is continued manually.

In the following, robot arms according to prior art are described.

DE 32 40 251 describes the control of a robot arm using an input device with an integrated force sensor. Here, the input device detects the forces and torques exerted by the user. These are transformed into an inertial system and the desired translation and rotation speeds of the robot end effector in the inertial system are then calculated on this basis.

DE 10 2018 114 644 B3 describes a robot manipulator which has a sensor between a distal robot link and an end effector for detecting a force winder, as well as a further sensor on the operating housing for detecting a user force or a user torque, respectively. Using the data from these two sensors, a computing unit uses a dynamic model of the robot to calculate a target force or target torque, respectively, for displacing the robot links and a target force or target torque, respectively, to be applied to an external object by the end effector.

DE 10 2013 019 869 A1 describes modular, manually operable input modules that can be mounted either between the arm modules, which are movable against each other, or on the tool interface of a robot arm. The user inputs on these modules can be used to control the axes of the robot arm located between the input module and the robot base and, for example, to teach a trajectory.

DE 10 2015 117 213 B4 describes an advantageous configuration of the last two axes of a robot arm. Here, the housing connected to the penultimate axis is cylindrical about the last axis. Optionally, this housing can be equipped with a graphic output unit. Above this housing, one or more input elements are located in a rotatable manner, in particular continuously with a detent or detachable clip connection, about the last axis. Below this housing is the tool interface connected to the last axis, which has a rigid extension for manual movement and at least one input element. The states of the input elements and the mechanical state of the robot arm, in particular positions and their derivatives, forces and torques, are used to generate the robot's control instructions.

DE 10 2016 222 675 A1 describes a robot operating hand-held device that can be coupled to a robot arm by means of a coupling device between the end effector and the robot arm. The robot operating hand-held device comprises a handle, several buttons and a force sensor, at the distal end of which the interface of the coupling device is located. This interface can be used to detachably connect the robot operating hand-held device to the coupling device on a robot arm. From this point onwards, the forces and torques exerted by the user on the handle can be measured and used to guide the robot manually.

EP 2 194 434 B1 describes the control of an industrial robot with the aid of a portable programming terminal and a manual guiding device, which is coupled to the movable robot structure or the end effector by means of quick coupling means. The manual guiding device can communicate wirelessly with the programming terminal assigned thereto. The programming terminal, in turn, is connected to the robot arm's control apparatus by cable or wirelessly. It is possible to configure the programming terminal or the control device in such a way that the movements of the robot are selectively limited for one or more degrees of freedom.

Takacs2020 (Takács, Bence, and Tamás Haidegger. “Autonomous applied robotics: Ultrasound-based robot-assisted needle insertion system concept and development.” 2020 IEEE 15th International Conference of System of Systems Engineering (SoSE). IEEE, 2020.) describes a system for vein injection consisting of an LBR iiwa robot (KUKA, Augsburg, Germany), an ultrasonic transducer and a tool with three degrees of freedom for positioning and inserting the injection needle. The interaction forces of the tool with the patient can be detected by means a force sensor between the tool and the tool interface of the robot.

In the Innomedicus Artemis system (https://www.innomedicus.com/de/urology/artemis/) for prostate biopsy, an ultrasonic transducer is mounted on a robotic arm and the ultrasound image is registered against preoperative image data. Parallel to the longitudinal axis of the ultrasonic transducer is a guide for the biopsy needle, which can thus be specifically aligned to previously planned points in the prostate.

Lim2019 (Lim, Sunghwan, et al. “Robotic transrectal ultrasound guided prostate biopsy.” IEEE Transactions on Biomedical Engineering 66.9 (2019): 2527-2537.) describes another robotic system for prostate biopsy, which however has similar characteristics and a similar workflow to the Innomedicus Artemis system.

Compared to the Innomedicus Artemis system, the iSR'obot™ Mona Lisa system (https://biobotsurgical.com/) for prostate biopsy also offers the possibility of changing the position and orientation of the biopsy needle with respect to the ultrasonic transducer. The translation movement of the biopsy needle is also actuated.

Welleweerd 2020 (Welleweerd, Marcel K., et al. “Design of an end-effector for robot-assisted ultrasound-guided breast biopsies.” International journal of computer assisted radiology and surgery 15.4 (2020): 681-690.) describes a system for ultrasound-guided breast biopsies. This system is configured as an end effector for a lightweight robot, which has an ultrasonic transducer, a stereo camera and a needle guide actuated by three servomotors.

JP 5637883 B2 describes a control device for a robot having a sensor on an operating handle.

A disadvantage of prior art systems and methods is that the control of robot arms, particularly as shown here, is imprecise.

The object of the disclosure is to provide a robot arm, an ultrasonic robot and a method for controlling a robot arm, wherein the control of the robot arm is optimized, in particular made more precise.

The object is achieved according to the disclosure with a robot arm, an ultrasonic robot, and a method for controlling a robot arm.

### SUMMARY

The robot arm according to the disclosure is in particular a medical robot arm. Particularly preferred, this is a hands-on robot arm. A hands-on robot arm in the context of the present disclosure is in particular a robot arm which is configured for direct interaction of a user with the robot arm, in particular with one or more robot links, for affecting the movements and/or the behavior of the robot arm. The robot arm has a robot structure with an arm link chain and a tool. The arm link chain has at least one arm link and at least one joint. The at least one joint is in particular a joint with one or two or three degrees of freedom. Preferably, the at least one joint is a Cardan joint or a ball joint. At least one tool is connected to the arm link chain, in particular distally. The tool is configured for connection, in particular detachable or non-detachable connection, to at least one end effector. The robot arm also has at least one end effector sensor for detecting forces and/or torques acting on an end effector connected to the tool. Preferably, one end effector sensor is provided for each end effector to be connected or connected. In particular, the end effector sensor is arranged and/or configured such that forces and/or torques acting on the end effector from the environment, in particular from a patient or a workpiece, are detected. The robot arm also has at least one structure sensor for detecting forces and/or torques acting on the robot structure. In particular, the structure sensor is arranged and/or configured such that forces and/or torques applied on the robot structure by a user are detected. The user is medical personnel in particular. Preferably, the arm link chain is connected proximally to a mounting device, e.g. a stand. Preferably, the robot arm, in particular the tool, has an input interface for user input. The input interface has, for example, at least one button, a thumbstick, a directional pad and/or a touchscreen. Preferably, the robot arm, in particular the tool, has an output interface for optical and/or acoustic output to a user. The output interface has, for example, at least one LED, a display and/or a loudspeaker.

The disclosure makes it particularly advantageous to be able to detect and quantify the physical contact of a user with the robot arm, in particular the arm structure. The present disclosure advantageously enables the detection of forces and/or torques acting on end effectors, in particular from the environment, such as a patient, and on the robot structure, in particular from a user. Preferably, a distinction can be made between forces on the end effector, in particular on the patient side, and forces on the tool and/or robot, in particular on the operator side, preferably by means of separate measurements. This represents a significant advantage over existing systems in which there is a pure position control of the robot system. This pure position control was found to be insufficient in the context of the disclosure. In contrast to the prior art, the disclosure thus advantageously also implements, in particular, permanent monitoring of the interaction forces between the robot system and the patient, e.g., in order to be able to detect deviations from the desired procedure at an early stage and/or to be able to correctly insert medical end effectors attached to the robot, e.g., ultrasonic transducers, biopsy forceps, injection needles.

The disclosure thus has particular advantages over most of the systems of the prior art, e.g. DE 10 2013 019 869 A1, DE 10 2015 117 213 B4, EP 2 194 434 B1, JP 5637883 B2, in which no distinction of the interaction forces between user and robot system and between robot system and patient or workpiece is possible. In DE 32 40 251, in particular in contrast to the disclosure, no dynamic decoupling of the components of the working space is possible. Since the robot structure in DE 32 40 251 itself does not have any sensors, in particular force-torque sensors, it is not possible to detect the interactions of a user. Compared to DE 10 2018 114 644 B3, there is, for example, the advantage that a specific configuration of the robot, in particular between the last axis and the tool interface, is not required and thus implementation is possible with common robots, in particular lightweight robots. In contrast to the present disclosure, the Takacs2020 telemanipulation system does not enable direct user interaction and therefore does not separately detect interaction forces with the environment and user interaction forces. In contrast to the Innomedicus Artemis system, Lim2019 and iSR'obot™ Mona Lisa system, the present disclosure particularly advantageously enables detection of the interaction of the end effector with the environment and the arm structure with a user. In particular, this also applies to the Welleweerd 2020 system, which is configured for autonomous operation.

Further advantages of the disclosure over the prior art, in particular as described above, are that the system of the present disclosure can be used with common robot arms, in particular lightweight robot arms, and/or that the system can be used for actuated end effectors.

In a preferred embodiment, the at least one end effector sensor is a force sensor, a torque sensor or a force-torque sensor. In particular, the force-torque sensor is a force-torque sensor with a degree of freedom of 2 to 6. On the other hand, it is also possible in particular that the end effector sensor is a force sensor with one degree of freedom, preferably in the longitudinal direction of the end effector. In a preferred embodiment, the at least one structure sensor is a force sensor, a torque sensor or a force-torque sensor. In particular, the force-torque sensor is a force-torque sensor with a degree of freedom of 2 to 6. A force-torque sensor with 6 degrees of freedom, also known as a 6-DOF force-torque sensor, is particularly preferred.

In a preferred embodiment, the arm link chain has several arm links and/or several joints. In particular, two arm links arranged next to each other are each movably connected to each other by a joint. The arm link chain preferably has several arm links with joints arranged between them.

In a preferred embodiment, a structure sensor is provided between the tool and the arm link chain for detecting forces and/or torques acting on the tool. This structure sensor is preferably a 6-DOF force/torque sensor. In particular, this structure sensor may be the only structure sensor of the robot arm according to the disclosure; on the other hand, at least one further structure sensor may preferably be provided for detecting forces and/or torques acting on the robot structure.

In a preferred embodiment, a structure sensor is provided for at least one arm link for detecting forces and/or torques acting on this arm link. Preferably, a structure sensor for detecting forces and/or torques acting on the respective arm link is provided for several arm links, particularly preferably for each arm link. In particular, this at least one structure sensor is connected to the respective arm link, preferably directly. Preferably, this at least one structure sensor is a 3-DOF force-torque sensor.

In a preferred embodiment, the robot arm has at least one end effector that is detachably or non-detachably connected to the tool. The at least one end effector is in particular an ultrasonic transducer, a biopsy forceps or an injection needle. In particular, several end effectors, preferably several of the above end effectors, are connected to the tool. In particular, the several end effectors are arranged in parallel. The at least one end effector is in particular configured to be movable or immovable, and/or actuated or non-actuated.

In a preferred embodiment, an end effector sensor for detecting forces and/or torques acting on the tool is arranged between the at least one end effector, in particular between each end effector, and the tool. Preferably, this at least one end effector sensor is connected to the respective end effector and the tool, preferably directly.

In a preferred embodiment, the robot structure has a tool interface arranged between the arm link chain and the tool for connecting the tool to the tool interface, in particular in a detachable manner. In particular, the tool interface has a tool center point (TCP). In particular, the tool center point is not a real part, but a point defined in relation to the last robot link. This point can also be located outside the tool, for example. This point is used in particular to describe the movements of the tool in Cartesian space.

In a preferred embodiment, a structure sensor is arranged between the arm link chain and the tool interface for detecting forces and/or torques acting on the tool interface, and thus in particular also on the tool. This structure sensor is preferably a 6-DOF force/torque sensor. In particular, this structure sensor may be the only structure sensor of the robot arm according to the disclosure; on the other hand, at least one further structure sensor may preferably be provided for detecting forces and/or torques acting on the robot structure.

In a preferred embodiment, the robot arm also has a control unit for controlling the robot arm. In particular, the control unit comprises a processor and/or a controller. The control unit is preferably configured to receive and, in particular, process the forces and/or torques detected by the end effector sensor and the structure sensor. Preferably, the control unit is configured to move the robot structure and/or to actuate the at least one end effector on the basis of the forces and/or torques received from and in particular processed by the end effector sensor and the structure sensor.

In a preferred embodiment, the robot structure has at least one virtual fixture, in particular for restricting and/or damping the movement of the robot arm. Preferably, the control unit is configured to adjust the at least one virtual fixture on the basis of the forces and/or torques received from and in particular processed by the end effector sensor and the structure sensor. In particular, the virtual fixture is not a real part. In particular, it is defined and parameterized in the software of the robot arm, preferably the control unit, in order to influence the behavior of the real robot arm, for example by preventing movements in certain areas.

The ultrasonic robot according to the disclosure is in particular an ultrasonic robot for facet joint injection. The ultrasonic robot comprises a robot arm with one or more features of the robot arm according to the disclosure. The ultrasonic robot also comprises an ultrasonic transducer connected to the tool as an end effector. It is preferable that the ultrasonic robot also has an injection needle connected to the tool as a further end effector. The ultrasonic transducer and injection needle are arranged in parallel, in particular. The injection needle is preferably arranged in the image plane of the ultrasonic transducer, but can in particular be moved and tilted therein.

One example in which the disclosure can be used to particular advantage is robot-assisted treatment of facet joint inflammation using ultrasound imaging. This generally involves injecting pain-relieving and anti-inflammatory medication into the facet joints of the human spine, using a live ultrasound image to position the needle correctly. Until now, such procedures have generally been performed manually, which places considerable coordinative and cognitive demands on the medical practitioner performing the procedure. The medical practitioner has to guide the ultrasonic transducer, interpret the ultrasound image and coordinate the injection needle at the same time. The mounting of the ultrasonic transducer and the needle guide to a robot arm enables a reproducible, gravity-compensated movement of the ultrasonic transducer (and, if desired, a limitation of the movement range) and at the same time ensures the correct positioning of the needle guide in relation to the ultrasonic transducer. It was found that the quality of the ultrasound image strongly depends on the adequate orientation of the transducer and the contact pressure. It is therefore advantageous to be able to measure the interaction forces between the ultrasonic transducer and the patient in the robot-assisted scenario. If the medical practitioner controls the system during the procedure by means of physical human-robot interaction, it can be implemented in an advantageous manner that the forces and torques exerted by the medical practitioner do not affect the measurement of the interaction forces.

The method according to the disclosure for controlling a robot arm is in, particular, a method for controlling a medical robot arm. Preferably, it is a method for controlling a robot arm with one or more features of the robot arm according to the disclosure or the ultrasonic robot according to the disclosure. The method relates in particular to the movement of the robot arm, preferably the robot structure. The method preferably relates exclusively to the movement of the robot arm, preferably the robot structure. In particular, the method does not relate to the control, preferably movement and/or actuation, of end effectors. Preferably, however, the method relates, for example, to the detection of forces during end effector actuation, e.g. during needle feed. Thus, the method can preferably detect a collision, e.g., with a bone, and react accordingly. For example, in doing so, the feed can be reduced or stopped, in particular, or a reverse feed can be performed, preferably by a predefined distance. The method comprises the step of detecting forces and/or torques acting on at least one end effector of the robot arm. The step of detecting forces and/or torques acting on at least one end effector of the robot arm is preferably carried out by means of at least one end effector sensor, preferably connected to at least one end effector. The method further comprises the step of detecting forces and/or torques acting on a robot structure of the robot arm. The step of detecting forces and/or torques acting on the robot structure of the robot arm is preferably carried out by means of at least one structure sensor, preferably connected to the robot structure. These two detection steps are preferably carried out at least partially simultaneously or successively. Preferably, the method further comprises the step of determining, in particular calculating, interaction forces and/or interaction torques of the robot arm using the detected forces and/or torques. The interaction forces and/or interaction torques are determined in particular by means of a control unit of the robot arm.

In a preferred embodiment, the method comprises the further step of outputting the interaction forces and/or interaction torques, in particular to an overall control of the robot arm. The “output” of the interaction forces and/or interaction torques is in particular a transmission of the interaction forces and/or interaction torques, and/or a visual display of the interaction forces and/or interaction torques.

In particular, the method further comprises storing the interaction forces and/or interaction torques, e.g. for documentation purposes. The method can also include further use of the detected forces and/or torques in the system control. For example, the robot movement, e.g., the TCP of the robot, can be regulated in certain Cartesian spatial directions based on the end effector forces and/or torques and/or in other Cartesian spatial directions based on the interaction forces and/or torques between the user and the tool or the user and the robot arm. In particular, it is also possible to use both pieces of information, wherein these are preferably weighted against each other using either fixed weighting factors or customizable weighting functions. In particular, it is also possible for signals to be processed before being passed on to the regulation, e.g., by signal filtering or adapting the signal frequency to the regulation frequency via downsampling and/or interpolation.

In the following, the disclosure is described in more detail by means of preferred embodiments with reference to the accompanying drawings.

### BRIEF DESCRIPTION OF THE DRAWINGS

The Figures show:

FIG. 1 a schematic illustration of an embodiment of an ultrasonic robot according to the disclosure with an embodiment of a robot arm according to the disclosure,

FIG. 2 a schematic illustration of a further embodiment of an ultrasonic robot according to the disclosure with a further embodiment of a robot arm according to the disclosure,

FIG. 3 a diagram representation of an embodiment of a method according to the disclosure for controlling a robot arm,

FIG. 4 a schematic illustration of a robotic system for visualizing an embodiment of a robot arm according to the disclosure and for visualizing a method according to the disclosure for controlling a robot arm,

FIGS. 5a-5b network views of teleoperation models for implementing an embodiment of an ultrasonic robot according to the disclosure, an embodiment of a robot arm according to the disclosure, and/or a method according to the disclosure for controlling a robot arm,

FIG. 6 a diagram representation of a strategy for generating the controller inputs for implementing an embodiment of an ultrasonic robot according to the disclosure, an embodiment of a robot arm according to the disclosure, and/or a method according to the disclosure for controlling a robot arm,

FIG. 7 a schematic, perspective view of a tool with end effectors for implementation in the context of a further embodiment of an ultrasonic robot according to the disclosure and/or a further embodiment of a robot arm according to the disclosure,

FIGS. 8a-8b illustrations of a tool with an end effector for implementation in the context of a further embodiment of an ultrasonic robot according to the disclosure and/or a further embodiment of a robot arm according to the disclosure,

FIG. 9 an illustration of kinematics for implementing an embodiment of an ultrasonic robot according to the disclosure, an embodiment of a robot arm according to the disclosure, and/or a method according to the disclosure for controlling a robot arm,

FIG. 10 an illustration of a further tool variant for implementation in the context of a further embodiment of an ultrasonic robot according to the disclosure and/or a further embodiment of a robot arm according to the disclosure,

FIGS. 11a-11b illustrations of a further tool variant for implementation in the context of a further embodiment of an ultrasonic robot according to the disclosure and/or a further embodiment of a robot arm according to the disclosure, and

FIGS. 12a-12b illustrations of a further tool variant with an end effector in the context of a further embodiment of an ultrasonic robot according to the disclosure and/or a further embodiment of a robot arm according to the disclosure.

### DETAILED DESCRIPTION OF THE PREFERRED EMBODIMENT

In the Figures, similar or identical components or elements are identified by the same reference numerals or variations thereof (e.g., 24 and 24ᵢ, 24ᵢ₊₁). In particular in the interest of improved clarity, preferably elements already identified are not provided with reference numerals in all Figures. The device features described can be implemented in particular in the context of ultrasonic robot 100 according to the disclosure and/or in the context of robot arm 10 according to the disclosure. The method features described can in turn be used in particular when carrying out the method according to the disclosure. Furthermore, the method features described can be implemented in particular in the context of the ultrasonic robot 100 according to the disclosure and/or in the context of robot arm 10 according to the disclosure, wherein preferably ultrasonic robot 100 and/or robot arm 10, particularly preferably a control unit 23 of ultrasonic robot 100 and/or robot arm 10, is configured to carry out at least one of the method features.

The concepts of the disclosure, in particular of the embodiments, are applicable in the field of hardware as well as in medical applications, in particular in medical applications in which a rotationally symmetrical tool is to be positioned with the assistance of a robot under intraoperative imaging, e.g. placement of bone screws, removal of biopsies. The methods, in particular regulation concepts, are also preferably transferable to other applications, e.g., in which the interaction forces between the user and the robot arm and between the robot arm and the environment are to be measured synchronously.

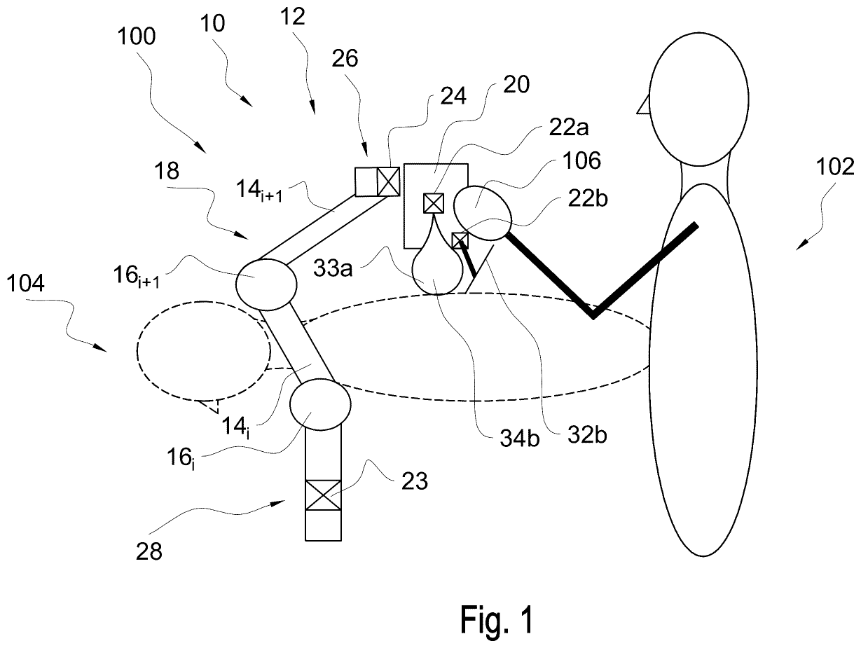

FIG. 1 shows an embodiment of an ultrasonic robot 100 according to the disclosure, in particular for facet joint injection, with an embodiment of a robot arm 10 according to the disclosure.

Robot arm 10 has a robot structure 12 with an arm link chain 18 and a tool 20 connected distally to arm link chain 18 via a tool interface 26.

Arm link chain 18 has two arm links 14ₗ, 14ₗ₊₁ movably connected by means of a joint 16ₗ₊₁. Arm link 14ₗ is movably connected to a stand 28 by means of a joint 16ₗ. A tool interface 26 is connected distally to arm link 14ᵢ₊₁.

A tool 20 is connected, in particular detachably, to tool interface 26. Tool interface 26 comprises a structure sensor 24. Structure sensor 24 is arranged in particular between tool interface 26 and tool 20 and/or between tool interface 26 and arm link chain 18. Structure sensor 24 is configured and/or arranged for detecting forces and/or torques applied by a user 102 to tool 20. In particular, structure sensor 24 is a 6-DOF force-torque sensor. In particular, user 102 is a medical personnel who, for example, moves tool 20 with hand 106 and thus applies forces and/or torques to tool 20.

Two end effectors 32a, 32b are connected, in particular detachably, to tool 20. End effector 32a is an ultrasonic transducer 32a, end effector 32b is an injection needle 32b. Injection needle 32b is connected to tool 20 by means of a needle guide 34b, which is adapted to be actuated and/or displaced in particular.

Two end effector sensors 22a, 22b are provided for detecting forces and/or torques from the environment, in particular from patients 104, for example during an ultrasound-assisted facet joint injection, on the end effectors 32a, 32b. End effector sensor 22a is configured and/or arranged to detect forces and/or torques acting on ultrasonic transducer 32a. In particular, end effector sensor 22a is arranged between ultrasonic transducer 32a and tool 20. End effector sensor 22b is configured and/or arranged to detect forces and/or torques acting on ultrasonic transducer 32b. In particular, end effector sensor 22b is arranged between injection needle 32b and tool 20, preferably between needle guide 34b and tool 20. End effector sensor 22a and/or end effector sensor 22b is in particular a force sensor, a torque sensor or a force-torque sensor with two to six degrees of freedom, particularly preferably a 6-DOF force-torque sensor. On the other hand, end-effector sensor 22a and/or end-effector sensor 22b can also be a force sensor with 1-DOF. Preferably, end effector sensor 22b is a 1-DOF force sensor, which in particular detects the force in the longitudinal direction of injection needle 32b.

The detected forces and/or torques acting on tool 20 of user 102 and on the end effectors 32a, 32b of patient 104 are detected by the sensors 24, 22a, 22b and can be used to control robot arm 10. In particular, the detected forces and/or torques are transmitted to a control unit 23 for robot arm 10. Interaction forces and/or interaction torques of robot arm 10 can be calculated from the detected forces and/or torques, preferably by control unit 23, and these can preferably be included in the overall control of the robot arm 10.

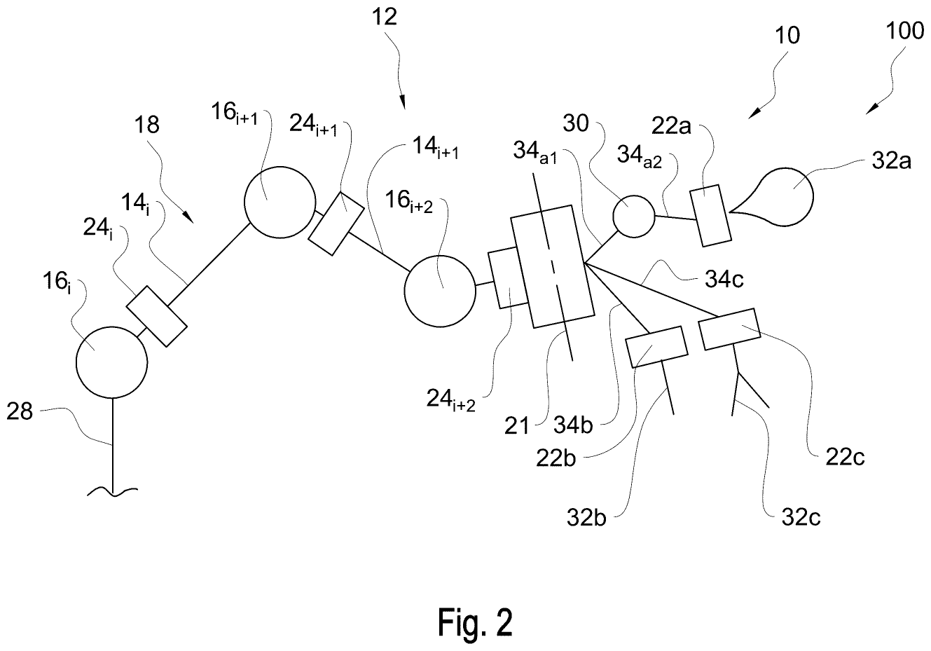

FIG. 2 shows a further embodiment of an ultrasonic robot 100 according to the disclosure, in particular for facet joint injection, with a further embodiment of a robot arm 10 according to the disclosure.

In the embodiment of FIG. 2, a structure sensor 24ᵢ, 24ᵢ₊₁ is provided for each arm link 14ᵢ, 14ᵢ₊₁, in particular connected to the respective arm link 14ᵢ, 14ᵢ₊₁.

Furthermore, in particular a structure sensor 24ᵢ₊₂ for detecting forces and/or torques on tool 20 is provided, in particular connected to tool interface 26.

The plurality of structure sensors 24ᵢ, 24ᵢ₊₁, 24ᵢ₊₂ are used for detecting forces, preferably of a user 102, on the respective elements of robot structure 12 in a particularly advantageous manner.

Three end effectors 32a, 32b, 32c are shown connected, in particular detachably, to tool 20. The illustrated end effector 32a is an ultrasonic transducer 32a, which is connected to tool 20 by means of two mounting elements 34a1, 34a2 that are movably connected by joint 30. Furthermore, an injection needle 32b is connected to tool 20 by means of needle guide 34b and a biopsy forceps 32c is connected to tool 20 by means of mounting element 34c as end effectors 32b, 32c. For each end effector 32a, 32b, 32c, an end effector sensor 22a, 22b, 22c is provided for detecting forces and/or torques on the respective end effector 32a, 32b, 32c, in particular arranged between end effector 32a, 32b, 32c and the tool.

Preferably, the following method steps can be implemented and/or the robot arm 10 can be configured of the following method steps: The interaction forces of a user 102 and robot structure 12 of robot arm 10 between two sensors 24ₗ, 24ᵢ₊₁ can be calculated by subtracting the measured values of two consecutive sensors 24ᵢ, 24ᵢ₊₁ in robot structure 12, taking into account the dynamics model of robot arm 10. The interaction forces of user 102 and tool 20 can be calculated as the difference between the measured values of the sensor of tool interface 26 and the sum of the measured values of the sensors 22a, 22b, 22c on the end effectors 32a, 32b, 32c, taking into account the dynamic model of tool 20; alternatively, an external force sensor between tool interface 26 and tool 20 could also be used in particular.

The interaction forces between the end effectors 32a, 32b, 32c and the environment can be detected, in particular read, directly using the respective end effector sensors 22a, 22b, 22c. In particular, force sensor 26 can be integrated into robot arm 10, preferably arranged between arm link chain 18 and tool interface 26, for example as shown in FIG. 2. With this arrangement, it is preferable that the weight and, in the case of dynamic movements, the mass inertia of tool interface 26 is determined and, in particular, included in the calculations of the tool interaction forces. Alternatively, the force sensor can be arranged in particular between tool interface 26 and tool 20.

With reference to FIG. 2, a preferred embodiment of the method according to the disclosure for controlling a robot arm is described below. However, the features described in the context of the following method can preferably also be implemented independently of the embodiment of FIG. 2 in the context of a method according to the disclosure for controlling a robot arm and/or in the context of a control unit 23 of a robot arm 10 according to the disclosure and/or in the context of a control unit 23 of an ultrasonic robot 100 according to the disclosure. If the steps are implemented within the context of a control unit 23, it is preferred that control unit 23 is configured, for example by means of processors and/or FPGAS, to execute corresponding method steps.

A first step involves detecting forces and/or torques acting on the end effectors 32a, 32b, 32c of robot arm 10, in particular by means of the corresponding end effector sensors 22a, 22b, 22c.

A second step involves detecting forces and/or torques acting on robot structure 12, in particular on the arm links 14ᵢ, 14ᵢ₊₁ and/or tool 20 of robot arm 10, in particular by means of the corresponding structure sensors 24ᵢ, 24ᵢ₊₁, 24ᵢ₊₂.

In particular, the first and second steps can occur in the following order: first step 1, then step 2; or first step 2, then step 1; or step 1 and step 2 in parallel; or step 1 and step 2 overlapping.

A further step involves determining, preferably calculating, interaction forces and/or interaction torques of the robot arm using the detected forces and/or torques, in particular by the structure sensors 24ᵢ, 24ᵢ₊₁, 24ᵢ₊₂ and/or the end effector sensors 22a, 22b, 22c. The interaction forces and/or interaction torques are determined in particular by means of a control unit 23 (cf. FIG. 1) of robot arm 10. Preferably, 6×1 force-torque vectors are calculated from the detected sensor data, wherein in particular forces and torques due to gravity and system dynamics are not taken into account.

A preferred calculation procedure of the method and/or the control unit of robot arm 10 is:

F_{User_ArmStructure_I}=F_{Sensor_ArmStructure_I}−F_{Sensor_ArmStructure_I+1}−F_{Dyn_ArmStructure_I}

with F_{Dyn_ArmStructure_I}: weight force and forces resulting from movement of the arm link i

F_{User_Tool}=F_{Sensor_TCP}−ΣₗⁿF_{Sensor_EndEffector1}−F_{Dyn_ToolStructure}

with F_{Dyn_ToolStructure}: weight force and forces resulting from movement of the tool with n: number of end effectors
F_{EndEffectorI}=F_{Sensor_EndEffectorI}
F_{EndEffectorII}=F_{Sensor_EndEffectorII}
F_{EndEffectorIII}=F_{Sensor_EndEffectorIII}
   wherein F_{User_ArmStructureI_I} is the external force-torque vector on robot structure 12 between joint 16; and joint 16ᵢ₊₁, in particular between the two structure sensors 24ᵢ, 24ᵢ₊₁, F_{Sensor_ArmStructureI} is the force-torque vector measured on joint 16ᵢ, in particular on structure sensor 24ᵢ, F_{User_Tool} is the external force-torque vector on tool 20, F_{Sensor_TCP} is the force-torque vector measured on structure sensor 24ᵢ₊₂, on tool interface 26 (TCP) of the robot arm, F_{Sensor_EndEffectorI} is the force-torque vector measured respectively on the end effector 32a, 32b, 32c with the respective end effector sensor 22a, 22b, 22c, and F_{EndEffectorI} is the external force-torque vector on the respective end effector 32a, 32b, 32c (cf. FIG. 2). The reference system can be body-fixed to the end effector or a fixed (inertial) world coordinate system, although it is preferable that all force-torque vectors are in the same reference system or are converted into it. The torques for the individual joints 16ᵢ, 16ᵢ₊₁, 16ᵢ₊₂ can be calculated in particular from the Cartesian force-torque vectors by multiplication with the associated Jacobian matrices, preferably body-fixed or inertial. In particular, the variables F are six-dimensional vectors with three forces and three torques.

Information on the preferred calculation procedure can be found in: Iskandar, Maged and Eiberger, Oliver and Albu-Schäffer, Alin and De Luca, Alessandro and Dietrich, Alexander (2021) Collision Detection, Identification, and Localization on the DLR SARA Robot with Sensing Redundancy; In: 2021 IEEE International Conference on Robotics and Automation, ICRA 2021. In particular, it is preferred in the context of the present disclosure to implement the calculation procedure from this source, wherein it is particularly preferred in the context of the disclosure to implement the method described therein as to how the second derivation of the measured joint position—i.e. the calculation of the acceleration—can be avoided in order to improve the signal-to-noise ratio.

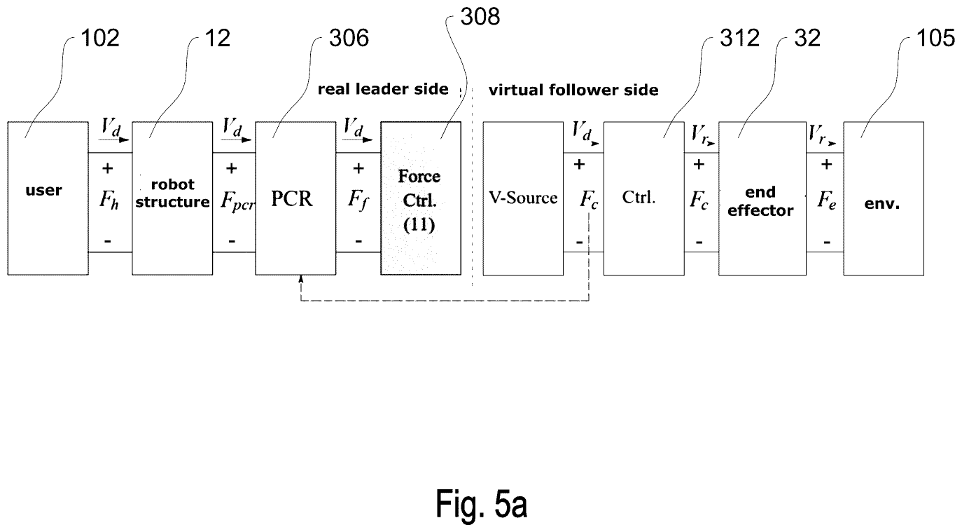

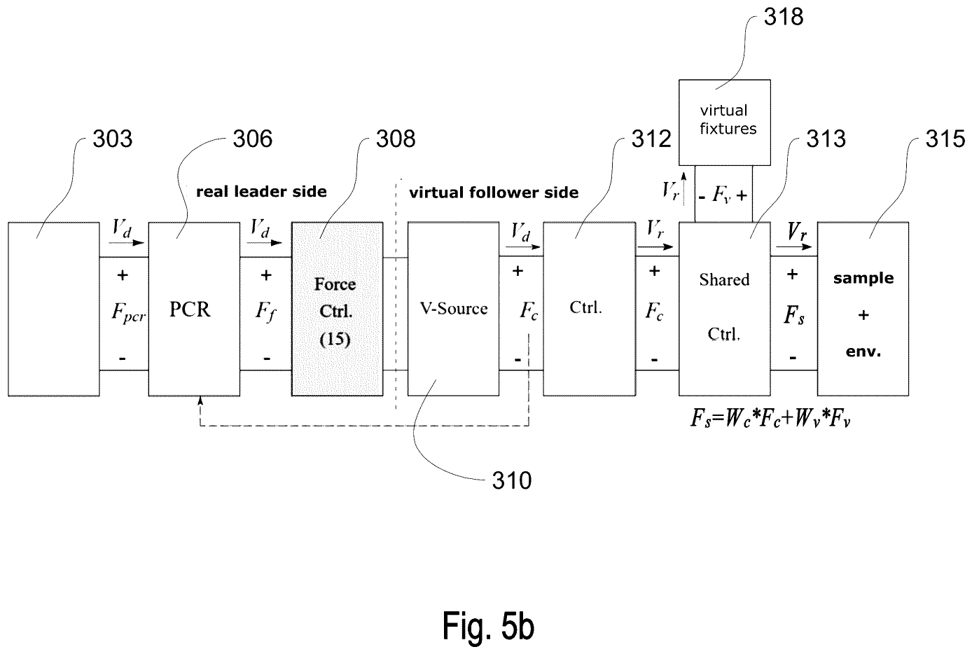

The calculated 6×1 force-torque vectors can be used in particular to regulate robot arm 10, preferably different joints 16ₗ, 16ₗ₊₁, 16ₗ₊₂ of robot arm 10. For example, F_{User_ArmStructureI} can be used to regulate the zero-space movement and F_{User_Tool} can be used to move tool 20 on tool interface 26 of robot arm 10, wherein virtual fixtures 318 (cf. FIG. 5b) based on F_{EndEffectorI} can optionally limit the interaction forces with the environment.

A preferred regulation architecture of the method and/or the control unit consists of a regulation architecture with decoupled degrees of freedom. In particular, a task space controller TSC(x [symbol] d vww [symbol] d) can be configured separately as part of the method and/or the control unit in each task space coordinate x [symbol] =(x, y, z, α, β, γ)^{T}. In this way, either a Cartesian impedance controller I, which controls positions and orientations with the desired interaction dynamics (mass-spring-damper), or a force/torque controller F, which controls the forces and torques, is activated independently for all coordinates of the task space. In particular, the control processes in different coordinates are dynamically decoupled in order to avoid crosstalk between the control processes in the different coordinates. For this purpose, a separate controller is in particular implemented for each of the six degrees of freedom.

A preferred strategy for generating controller inputs of the method and/or the control unit 23 is: The desired inputs x [symbol] d=(xd, yd, zd, αd, βd, γd)T for the impedance controller I or ww [symbol] d=(ffx,d, ffy,d, ffz,d, τx,d, τy,d, τz,d)T for the force/torque controller F are provided in particular by the high-level software of the robot system. If this only provides discrete target values, a Cartesian interpolator is used in particular to provide the desired states at the control rate. If the high-level software cannot provide a suitable input for a coordinate jj, the controller is switched to Fjj=0 in particular in order to control a zero force or a zero torque for this particular coordinate jj. This means that the robot can be driven backwards in this coordinate. Since no forces are applied, the control is inherently safe. This allows the user to intervene by manually correcting or terminating the respective subtask in hands-on mode.

Preferably, it is implemented in the method and/or the control unit 23 is configured, in particular by means of high-level software, to identify the current step in the operation procedure using the system status and the sensor data and to adjust the controller inputs based thereon. If the current operation step cannot be determined with sufficient certainty, either a user input can be requested or the strategy described above for generating the controller input without a suitable input comes into effect. To ensure that sufficiently accurate statements can be made about the reliability of the determination of the operation step, the associated error estimates are preferably also reported in addition to the sensor values, preferably to control unit 23, and particularly preferably to the high-level software.

A combination of inertial scaling, haptic guidance with virtual fixtures and force feedback in control unit 23, for example in a controller of control unit 23 or in the context of the method, is also preferred. Preferably, this enables not only precise feedback of the measured forces to user 102, in particular by means of the proposed controller, but also modification of the measured values by inertial scaling and virtual fixtures.

A preferred strategy for optimizing back-driveability by inertial scaling of the method and/or control unit 23 is: In order to increase the back-driveability even further, inertial scaling can preferably be implemented; in particular, the controllers can include inertial scaling. In particular, this modifies the inertia perceived by user 102 according to the requirements of the respective application. In hands-on mode, for example, this can give the user the feeling that the robot arm they are moving is, in particular virtually, massless and/or frictionless. Preferably, for this purpose, the weight of the rest of the tool is compensated for, so that preferably the user has the feeling of only moving the ultrasonic transducer.

Preferably, virtual fixtures are implemented in the context of the method and/or by means of control unit 23: During manual movement of robot arm 10, virtual fixtures AVirtualFixture(x [symbol] d, pVF,d) in particular can be activated and parameterized by pVF,d in particular to haptically represent guidance primitives and exclusion zone restrictions, which can reduce the cognitive load on the user and increase safety. In particular, the virtual forces FV resulting from the virtual fixtures can be added to the pre-processed force-torque vectors on the end effectors F_{EndEffectorI} in order to realize virtual haptic guidance.

Preferably, disturbance observers and/or safety systems are implemented in the context of the method and/or by means of control unit 23: Preferably, at least one disturbance observer PDistObs(ww [symbol] EE) monitors all F/T sensor information of robot arm 10, in particular permanently, in order to distinguish between unintentional collisions and intended user or environmental interactions. Safety is guaranteed in particular by limiting forces and velocities and ensuring the stability of the regulated system, e.g. by means of passivity observers.

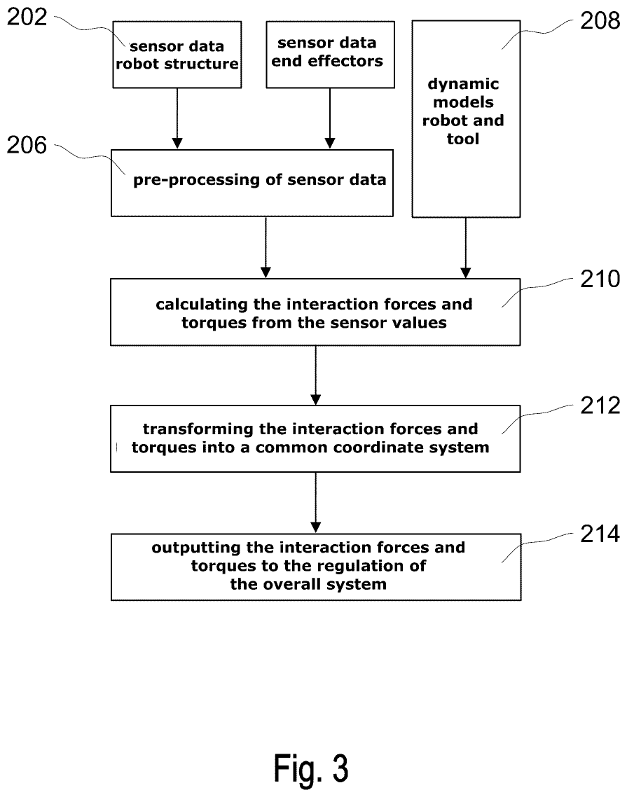

A preferred embodiment of the method according to the disclosure is shown in FIG. 3 in a flow chart. First, forces and/or torques of robot structure 12 are detected, in particular by means of at least one structure sensor 24 (step 202), and forces and/or torques of the at least one end effector 32 are detected, in particular by means of at least one end effector sensor 22 (step 204).

Preferably, detected forces and/or torques are pre-processed, for example by filtering noise and/or compensating for a static offset (step 206). Subsequently, in particular the pre-processed forces and/or torques together with the forces and/or torques resulting from a dynamic model of robot arm 10 (step 208) are used to calculate the interaction forces and/or torques (step 210). Preferably, these are then transformed into a common coordinate system (step 212) and preferably output to the control, in particular the regulation, of the overall system of robot arm 10 (step 214).

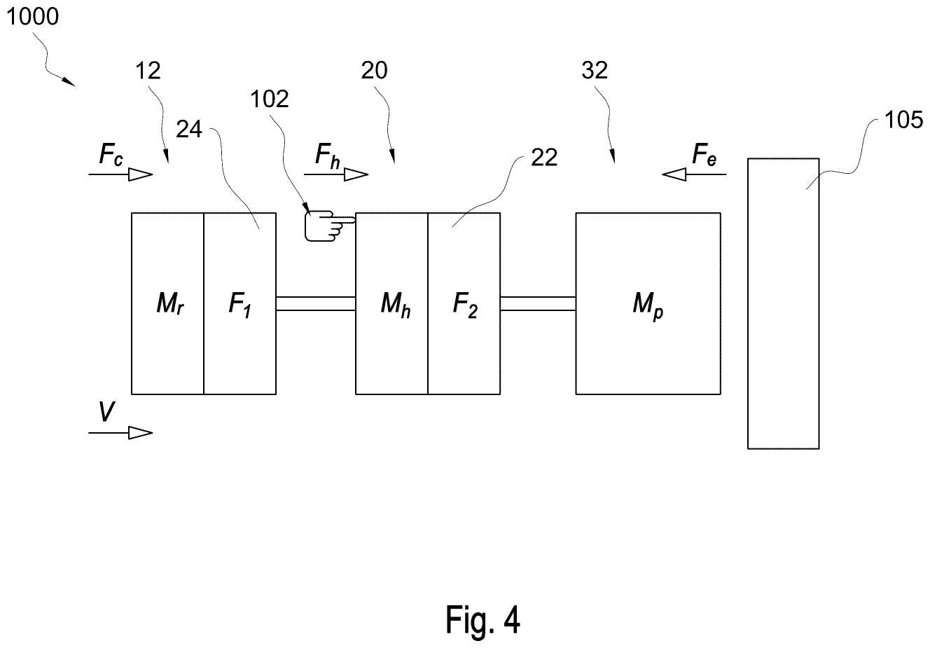

FIG. 4 shows a schematic illustration of a preferred robotic system 1000 for implementation in an ultrasonic robot 100 according to the disclosure and/or a robot arm 10 according to the disclosure. The forces F and masses M of the robotic system 1000 are shown in one degree of freedom. The illustrated system 1000 includes robot structure 12, tool 20 and end effector 32. The external force Fₕ acting on the system, e.g. by a user 102, and force Fₑ, by the environment 105, e.g. a patient 106, can be determined with the aid of the respective sensors 22, 24, in particular configured as 6-DOF force-torque sensors. F_{c} is the force set by a preferred force controller on robot arm 10 in this degree of freedom.

The two sensors 22, 24 provide, in particular, the forces F₁ and F₂ as measured values, wherein, preferably if the forces and/or torques as a result of the masses and mass inertias of the end effector and tool are not taken into account, the following applies:

F₂=−Fₑ

F₁=Fₕ−Fₑ

therefore

Fₑ=−F₂

Fₕ=F₁−F₂.

The equation of motion of the overall system is therefore as follows:

Mₜ\dot{V}=F_{c}+Fₕ−Fₑ−DV,

in which:

Mₜ=Mᵣ+Mₕ+Mₚ,
V is the velocity of the overall system, and
D the damping of the robot.

The actuating force of a force regulator for inertial scaling can be calculated in particular as F_{c}=K*(Fₕ−Fₑ).

This results in the following equation of motion for the overall system:

$$\begin{matrix}{M}_{i} \dot{V} = K ( {F}_{h} - {F}_{e} ) + {F}_{h} - {F}_{e} - DV , \\ {M}_{1} \dot{V} = ( 1 + K ) {F}_{h} - ( 1 - K ) {F}_{e} - DV , \frac{{M}_{i}}{1 + K} \dot{V} + {F}_{e} + \frac{DV}{1 + K} = {F}_{h}\end{matrix}$$

The last line describes the force Fₕ felt by the user when moving the tool. In particular, however, the user should ideally have the feeling of moving the end effector alone when moving the tool attached to the robot and feeling its interaction forces with the environment Fₑ:

Mₚ\dot{V}+Fₑ=Fₕ

By adjusting the scaling factor K of the force controller accordingly, the following behavior can almost be achieved:

$$\frac{{M}_{i}}{1 + K} = {M}_{p} , or K = \frac{{M}_{i}}{{M}_{p}} - 1$$

The remaining disturbance variable as a result of damping D can be further reduced by inserting a damping term in F_{c}.

FIGS. 5a and 5b show network views of teleoperation models for implementing an embodiment of an ultrasonic robot 100 according to the disclosure, an embodiment of a robot arm 10 according to the disclosure, and/or a method according to the disclosure for controlling a robot arm 10.

In particular, the system described above in FIG. 4 can be converted into the teleoperation model shown in FIG. 5a. The real leader comprises robot structure 12 and the virtual follower comprises end effector 32, in particular with the desired dynamics. The PCR 306 (Passive Coupling Reference) block ensures that the overall system behaves passively, i.e., does not generate any energy.

The port network representation uses velocity (V) and force sources (F). If user 102 moves robot 12 at a velocity Vd, this will be communicated in particular to the virtual follower, so that the virtual follower, in particular end effector 32, also moves at the desired velocity Vd. This is done in accordance with block “V-Source” 310. The desired velocity Vd is commanded to the Ctrl block, position controller 312, so that the Ctrl generates the force Fc to move end effector 32 to follow the velocity Vd by comparing it to the velocity of end effector Vr.

In particular, for shared control applications, virtual fixtures 318 are implemented in FIG. 5b to stop the movement of the end effector in certain directions by applying a force Fv to the end effector. End effector block 315 receives a weighted sum of the forces of Ctrl 312 and the virtual fixtures 318. This means that Fs=WcFc+WvFv, wherein the weights Wc and Wv determine the control authority of the forces Fc and Fv, in particular who has more control effect on end effector 32. For example, if Wv=0, end effector 32 is moved completely by the user. If, for example, Wc=0, the virtual fixtures have complete control over the movement of the end effector. The weights Wc and Wv can assume values between 0 and 1, depending on which degrees of freedom/movement directions need to be controlled by the two controllers. These weights are determined by block 313 of the common control.

FIG. 5b shows an extension of the teleoperation model from FIG. 5a with virtual fixtures 318. The forces generated by the virtual fixtures 318, in particular virtual forces, are applied to the virtual follower as additional forces and transmitted to user 102 by the force F_{f} generated by force controller 308 on the leader side, wherein the following applies: F_{f}=K*(Fₕ−Fₑ−Fᵥ).

User 102 and robot structure 12 are combined to block 303, end effector 32 and environment are combined to block 315.

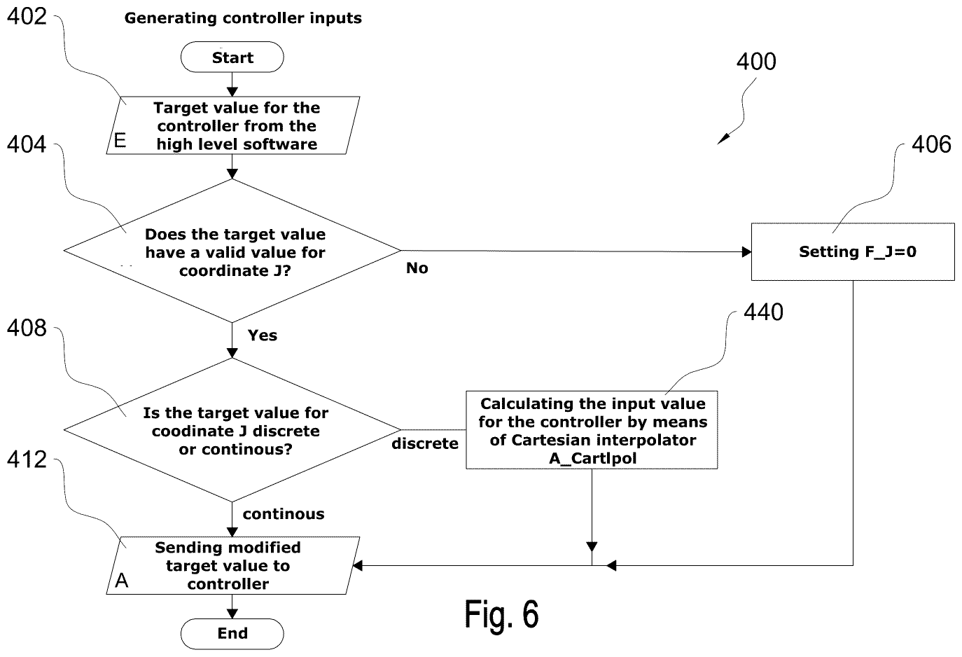

FIG. 6 shows a diagram representation of a strategy for generating the controller inputs for implementing an embodiment of an ultrasonic robot 100 according to the disclosure, an embodiment of a robot arm 10 according to the disclosure, and/or a method according to the disclosure for controlling a robot arm 10.

In order to ensure that the regulation always receives valid default values, the target values (step 402) for forces and/or torques, for example from control unit 23, in particular from high-level software, of robot arm 10 are checked again (step 404) and modified if necessary (step 406) before being passed on to the control, in particular the regulation. If the high-level software does not provide a valid target value for a coordinate j, a zero force is commanded in this coordinate. If control unit 23, in particular the high-level software, provides only a discrete value for a coordinate j, i.e. in particular with an update rate<control rate of the robotic system, the input value for the controller is calculated with the aid of a Cartesian interpolator for intervening time steps and modified accordingly (steps 410).

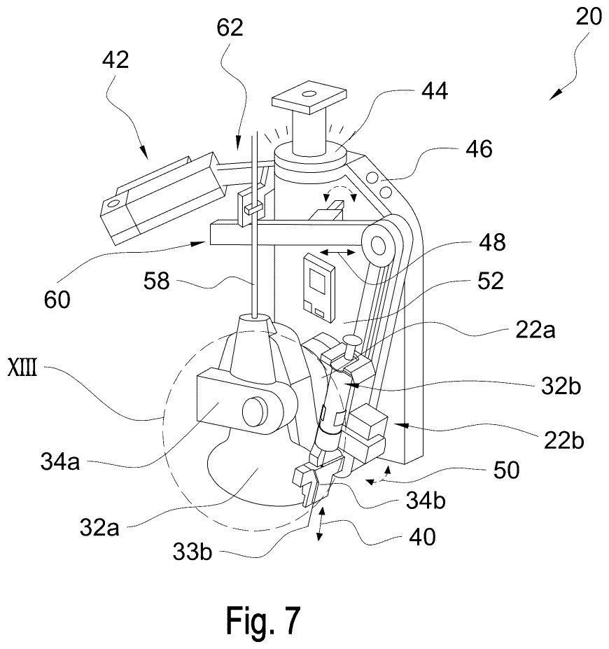

FIG. 7 shows schematic, perspective view of a tool 20 with end effectors 32a, 32b for implementation in the context of a further embodiment of an ultrasonic robot 100 according to the disclosure and/or a further embodiment of a robot arm 10 according to the disclosure. In particular, tool 20 is a tool for ultrasound-assisted facet joint injection.

In particular, this tool 20 has a modular structure to support the use of different ultrasonic transducers 32a and injection needles 32b.

Ultrasonic transducer 32a is configured in particular to provide intraoperative images, for example to correctly position the tip of injection needle 32b on the facet joints of the spine. Ultrasonic transducer 32a is detachably connected by the positive transducer mounting 34a to an end effector sensor 22a, in particular configured as a 6-DOF force-torque sensor, which can measure the interaction forces between patient 106 and ultrasonic transducer 32a. The proximal side of the force-torque sensor is connected to the central structural part 52 of tool 20.

Needle guide 34b is positioned and aligned by means of an actuated translational degree of freedom 48 and an actuated rotational degree of freedom 50. A further actuated translational degree of freedom 40 is provided for moving syringe 33b of injection needle 32b along its longitudinal axis, wherein a particularly optional end effector sensor 22b detects the interaction forces of injection needle 32b. Preferably, a stereo camera 42 can be used to monitor movements of patient 106 and provide an initial estimate for the positioning of ultrasonic transducer 32a. Preferably, integrated buttons 46 serve as input interface, and in particular a ring of RGB LEDs 44 as output interface for user 102.

In general, it is advantageous that the guide for injection needle 32b is positioned and aligned as soon as the facet joint to be treated is clearly recognizable in the ultrasound image. Injection needle 32a should point to the entrance to the facet joint, keep the path through the patient tissue as short as possible and pass sufficiently close to the center of the ultrasound image, especially since the image quality decreases towards the edge. Since injection needle 32a should pass in the image plane of the ultrasound image, this is a planar problem. Therefore, at least one degree of rotational freedom is preferred for the orientation of needle guide 34a in the image plane. In addition, a second degree of freedom is advantageous to allow repositioning of injection needle 32b without moving ultrasonic transducer 32a. This repositioning is possibly preferred if injection needle 32b deviates from its desired path during insertion due to the nature of the tissue and the planned injection site can therefore not be reached. A movement of ultrasonic transducer 32a should be avoided, as this can impair the registration between the preoperative planning and the ultrasound image and also contaminate the injection site of injection needle 32b with ultrasound gel.

Preferably, as shown in FIG. 7, tool 20 has a mounting option 62 for a stereo camera 60 or another, in particular similar, optical sensor. In particular, this sensor is mounted such that it can detect the three-dimensional skin structure of patient 106 in the vicinity of ultrasonic transducer 32b. In particular, the captured 3D surface can then be compared with the preoperative imaging data, e.g. typically CT images, in order to identify the approximate position of the facet joint to be treated. In autonomous operation, ultrasonic transducer 32b is then positioned close to the patient's spine, in particular based on this position estimate, which may preferably have errors due to the movement of the patient's soft tissue 106, among other things. Then, in particular ultrasound images of the spine in the vicinity of the estimated position are compared with the image data from the preoperative planning and registered. In particular, once ultrasonic transducer 32b is positioned such that its image section corresponds to the image from the preoperative planning, the needle guide can be positioned and aligned according to the planning.

The described approximate positioning of the ultrasonic transducer with the aid of stereo camera 42 can be carried out autonomously, i.e. without the intervention of user 102. On the other hand, it reduces the search space for the registration of ultrasound images and preoperative image data in particular. Since this registration is complex and computationally intensive due to the different imaging modalities, especially preoperatively usually CT images and intraoperatively ultrasound images, the risk of errors and the computational effort can be reduced.

Since user 102 in particular is to interact directly with tool 22, the latter preferably has a user interface suitable for this purpose, comprising input interface 46 and/or output interface 44.

Input interface 46 should in particular enable navigation through a simple menu structure, which is why buttons, e.g., up, down, confirmation/plane change, were integrated in the present tool 20. However, other interfaces such as thumbsticks, directional pads or a touchscreen can also be used.

Output interface 44 is in particular intended to provide user 102 with feedback on the current system status. In the present system, an RGB LED ring 44 is preferably provided for this purpose, particularly in the area of robot interface 46; other possible designs would be, for example, individual status LEDs or a small display.

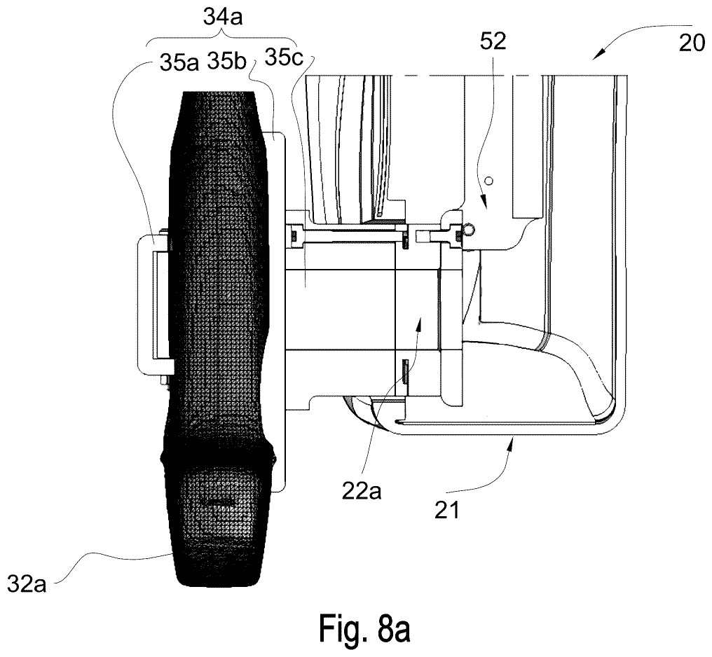

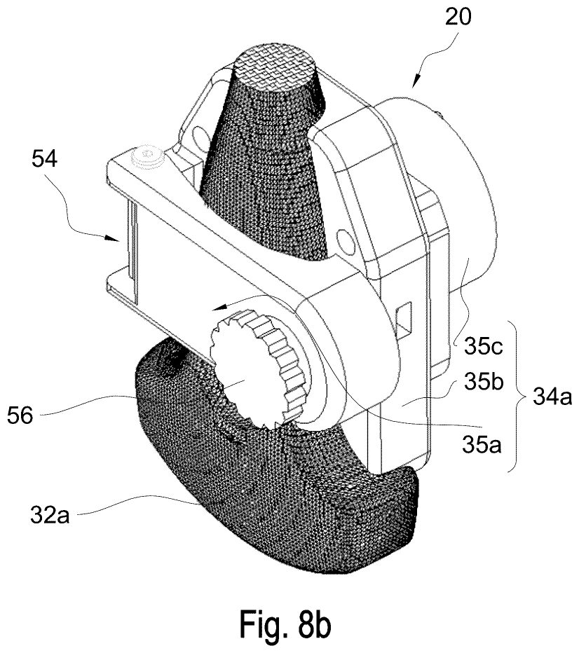

FIG. 8a shows a sectional view of a tool 20, in particular a partial tool 20, with an end effector 32b for implementation in the context of a further embodiment of an ultrasonic robot 100 according to the disclosure and/or a further embodiment of a robot arm 10 according to the disclosure. Preferably, the partial tool 20 of FIG. 8a is a detailed view of section VIII in FIG. 7.

FIG. 8b shows a perspective, lateral view of tool 20 of FIG. 8a, in particular with transducer 32a, mounting 34a and sensor 22a.

Ultrasonic transducer 32a is connected to the distal side of an end effector sensor 22a, in particular configured as a 6-DOF force-torque sensor, by means of a transducer mounting 34a with a form-fit and/or force-fit connector including the elements 35a, 35b and a mounting plate 35c. The proximal side of end effector sensor 22a is connected to the central structural part 52 of tool 20. In particular, housing 21 of tool 20 is configured such that it does not touch the distal sensor side in order not to impair the measurement. In particular, both latch 35a, which can be fixed by means of a hinge 64 and a knurled screw 56, and negative mold 35b are transducer-specific, while the other components of transducer mounting 34a in particular can be used for different models of ultrasonic transducers 32a.

Due to the preferred use of a separate mounting plate 35c, transducer mounting 34a can be replaced when ultrasonic transducer 32a is changed without having to loosen the screw connection on the actual sensor. Since assembly errors, e.g. incorrect screw length or excessive torque, can lead to damage to end effector sensor 22a with this screw connection, this reduces the risk of damage in particular. The attachment of the model-dependent transducer mounting 34a to mounting plate 34a is preferably detachable without tools, e.g., by means of clicking catches, magnets or knurled screws. Likewise, the locking mechanism of transducer mounting 34a is preferably adapted to be fixed without tools. If transducer mounting 34a is configured to be form-fit to ultrasonic transducer 32a, repeated insertion and removal of ultrasonic transducer 32a is possible after a single calibration between the transducer pose and transducer mounting 34a, without the need for calibration between the transducer pose and transducer mounting 34a each time.

In order to avoid disturbance forces of a cable 58 of ultrasonic transducer 32a (cf. FIG. 7), it is preferable that cable 58 is guided to the proximal side of end effector sensor 22a without tension and fixed there, for example by means of a clamp 60 (cf. FIG. 7). Since ultrasonic transducer 32a can only move minimally in transducer mounting 34a, the disturbance forces and torques that then occur due to cable 58 are, in particular, almost constant and can be easily taken into account when processing the sensor values.

Preferably, transducer mounting 34a is located outside housing 21 of tool 22. This allows ultrasonic transducer 22a to be attached and removed without opening housing 21. This prevents possible damage or contamination of the components inside the housing. In this case, it is preferable that there is an opening in housing 21 in the area of end effector sensor 22a in order to prevent housing 21 from interfering with the force measurement. To seal this opening against dirt and/or liquids, a seal is preferably to be provided which either functions without contact, e.g. labyrinth seal and/or has the lowest possible rigidity, e.g. silicone foam. This minimizes the disturbance forces and torques transmitted from the housing to end effector sensor 22a.

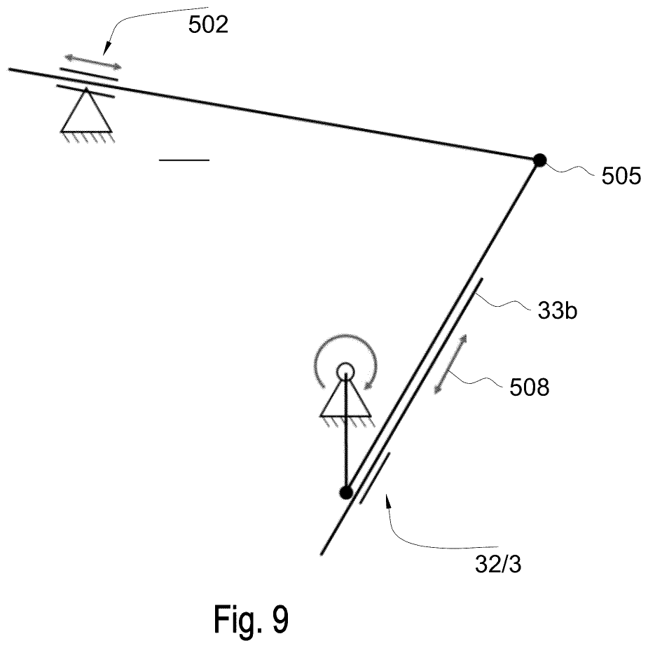

FIG. 9 shows a cinematics for implementing an embodiment of an ultrasonic robot 100 according to the disclosure, an embodiment of a robot arm 10 according to the disclosure, and/or a method according to the disclosure for controlling a robot arm 10. The kinematics according to FIG. 9 is used in particular for positioning and orienting the injection needle within the image plane of the US transducer.

Shown is a closed kinematic chain with two actuated degrees of freedom for positioning and alignment of needle guide 34b: Needle guide 34b is aligned in particular by a translationally activated degree of freedom 502 and a rotationally activated degree of freedom 503, which are coupled to one another by means of two passive joints 504, 505. In particular, the translational degree of freedom 502 adjusts the inclination of needle guide 34b, and the rotational degree of freedom 503 adjusts the distance of needle guide 34b to ultrasonic transducer 32a along a circular path. Since the rotational degree of freedom 503 preferably only performs small angular changes, needle guide 34b is predominantly moved horizontally by it and only minimally lifted from the skin of patient 106, wherein in particular the vertical movement is less than 10% of the horizontal movement for deflections of up to 10° due to the trigonometric relationships. In particular, needle 33b can be moved with respect to needle guide 34b by a separate translational degree of freedom 508.

Since a high rigidity of the needle guide is desirable, a closed kinematic chain with two driven degrees of freedom 502, 503 was selected in the embodiment of FIG. 9. Both degrees of freedom 502, 503 have a limit switch, particularly at one end of their range of movement, in order to enable an initialization run after tool 20 is switched on. The range of movement of needle guide 34b changes depending on the dimensioning of the actuated degrees of freedom 502, 503.

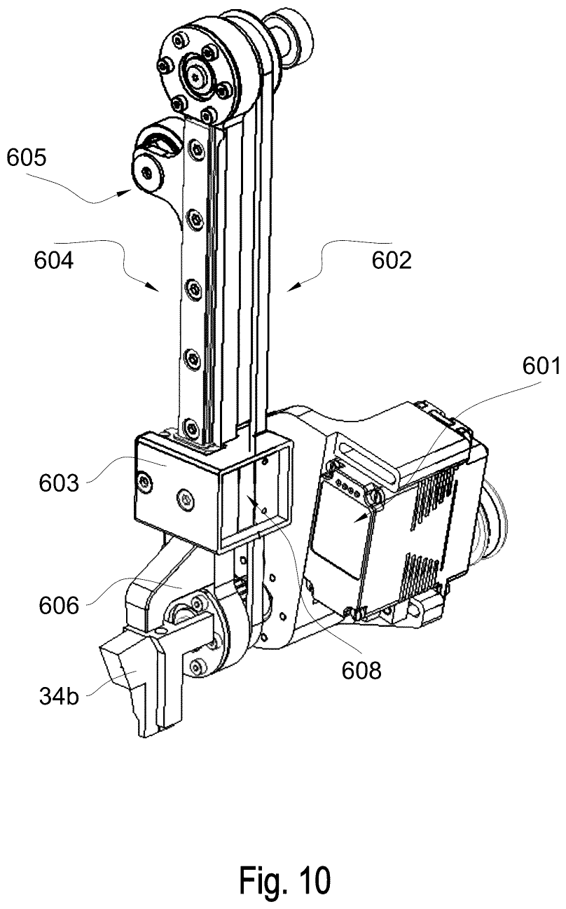

FIG. 10 shows an illustration of a further tool variant, in particular a partial tool 20, for implementation in the context of a further embodiment of an ultrasonic robot 100 according to the disclosure and/or a further embodiment of a robot arm 10 according to the disclosure.

A preferred motor 601 drives a toothed belt 602, which is positively connected to trolley 603 on linear rail 604. Toothed belt 602 is pre-tensioned in particular by means of an eccentric roller 605. An intermediate element 606 for fastening needle guide 34b is attached near the patient-side end of linear rail 604. In the illustrated configuration of trolley 603, it is equipped with a magnet 608 for attaching a syringe attachment.

Preferably, the planned tool 20 should also support autonomous performance of the facet joint injection. For this purpose, it is preferable that the degree of translational freedom for the needle feed is driven, e.g., by means of motor 601 as shown in FIG. 10.

In particular, a limit switch is located at one end of rail 604 to enable an initialization run after tool 20 is switched on. Injection syringe 32b is attached to trolley 603 in particular such that syringe needle 33b is aligned with needle guide 34b at the patient-side end of the profiled rail guide.

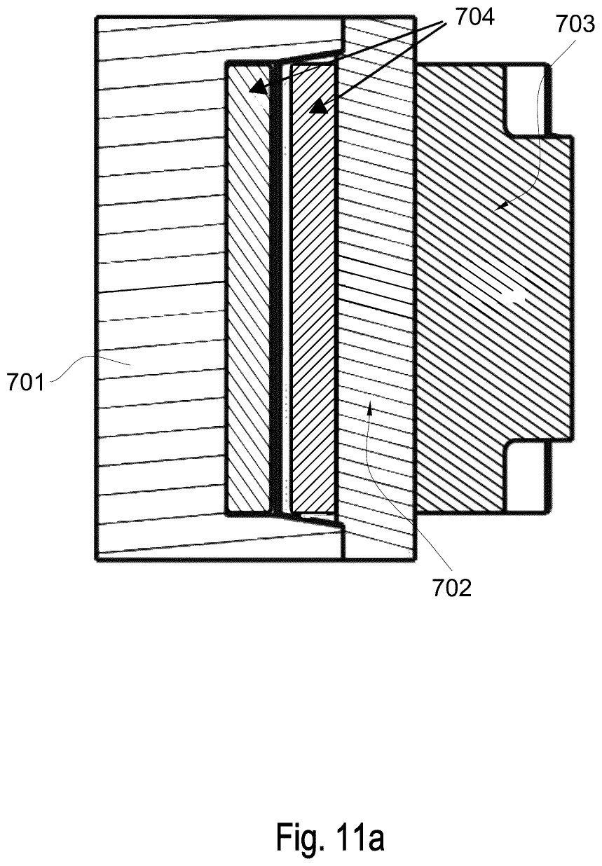

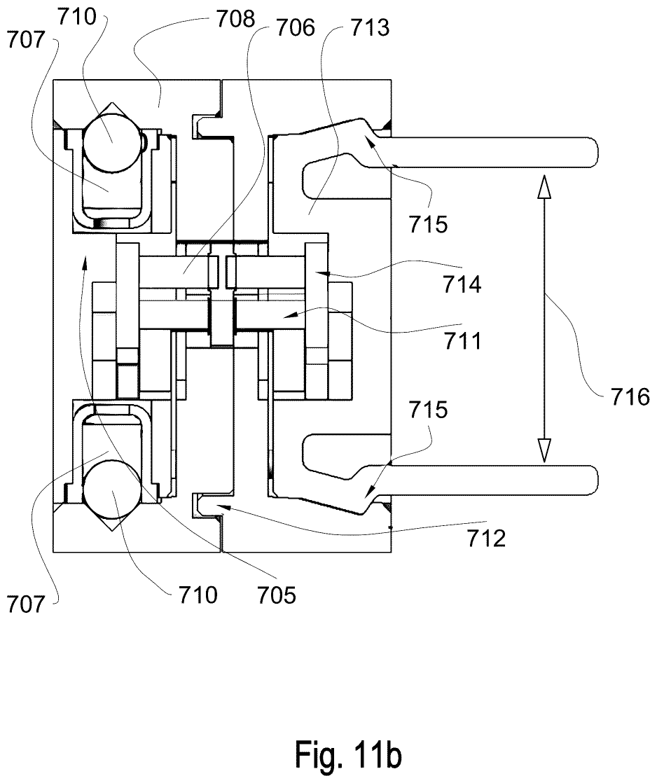

FIG. 11a shows a sectional view of a further tool variant, in particular a partial tool 20, for implementation in the context of a further embodiment of an ultrasonic robot 100 according to the disclosure and/or a further embodiment of a robot arm 10 according to the disclosure.

Preferably, particularly in the embodiment shown in FIG. 10, a separation plane is provided in the syringe attachment in order to enable sterile packing (“draping”) of tool 20 during the surgical procedure. Both the injection site of the injection needle and its surroundings must be kept sterile during a procedure, as must all system components that come into contact with it. The latter relates in particular to injection needle 32b and needle guide 34b. Since tool 20 and robot structure 12 cannot generally be sterilized due to the electronic components they include and their complex geometry, it is preferable to wrap them with a sterile drape during the surgical procedure. Preferably, ultrasonic transducer 32a must either not touch the injection site during the procedure or must also be draped in a sterile manner. Thus, it is preferred that injection needle 34a is in any case outside the sterile drape, while the rest of tool 20 is inside.

FIG. 11a shows a possible configuration, in particular based on the embodiment according to FIG. 10, wherein trolley 701 and syringe attachment with elements 702, 703 are configured separately, and wherein the parts are coupled by means of magnets 704. The sterile drape can be passed between the two magnets 704 without being damaged.

In an alternative configuration to the embodiment of FIG. 11a according to FIG. 11b, an intermediate element is inserted into the sterile drape in the area of the syringe attachment. This can be a plastic frame, for example. Depending on the configuration, tool 20, the intermediate element and the syringe attachment 34b can be connected by means of various mechanisms, preferably mechanisms that can be fixed and detached without tools, e.g., magnets, snap locks or screws. In particular, although this alternative solution is technically more complex, it is particularly advantageous if an electrical interface is also to be routed through the drape at this point.

A printed circuit board with several spring contact pins 706 and two resilient pressure pieces 707 are attached to the trolley of linear guide 705 or, for example, another component located inside the drape. When draping the system, the two-part plastic frame with elements 708, 709 fixed in an opening of the drape is first pushed onto trolley 701 until the sides of trolley 701, which are preferably slightly beveled, come into contact with the sides of the recess in the plastic frame, which are also preferably slightly beveled. This locks all degrees of freedom except horizontal translation. By engaging the balls of the resilient pressure pieces 710 in recesses in the plastic frame, this last degree of freedom is also locked. A printed circuit board with contact pads on both sides 711 is already fixed in advance inside the two-part plastic frame. The sterile drape was also fixed in advance in the tongue and groove connection 712 between the two frame parts. Now, syringe attachment 713 with the printed circuit board attached thereto can be pushed with several spring contact pins 714 in a horizontal direction into the right-hand recess of the plastic frame until the sides of trolley 701, which are preferably slightly beveled, come into contact with the sides of the recess in the plastic frame, which are also preferably slightly beveled. When the target position is reached, the catches 715 engage in the corresponding recesses in 719 and also block horizontal translation. The extensions 716 on the catches 715 make it easier to detach the syringe attachment from the plastic frame.

Preferably, the actuated mechanism for the needle feed is provided with an end effector sensor 22b, in particular configured as a force sensor, for measuring the force in the longitudinal direction of injection needle 32b. This allows unexpected tissue conditions, e.g., scar tissue, and collisions with the bone of the spine to be detected immediately and the application of excessive forces to be prevented. In particular, the sensor described has at least one degree of measurement freedom in the direction of the needle axis. However, sensors with more degrees of freedom can also be used, for example to detect lateral forces on the needle as a result of patient movements. End effector sensor 22b can be located either inside or outside the drape. When attached inside the drape, end effector sensor 22b is in particular not packaged sterilely anymore, but forces acting on the drape can impair the measurement. Such disturbances do not occur when the sensor is attached outside the drape, however, end effector sensor 22b should be sterile, in particular, and therefore either repeatedly sterilizable or a cost-effective disposable product. In this case, in particular, the power and communication lines of end effector sensor 22b should also be routed through the sterile barrier.

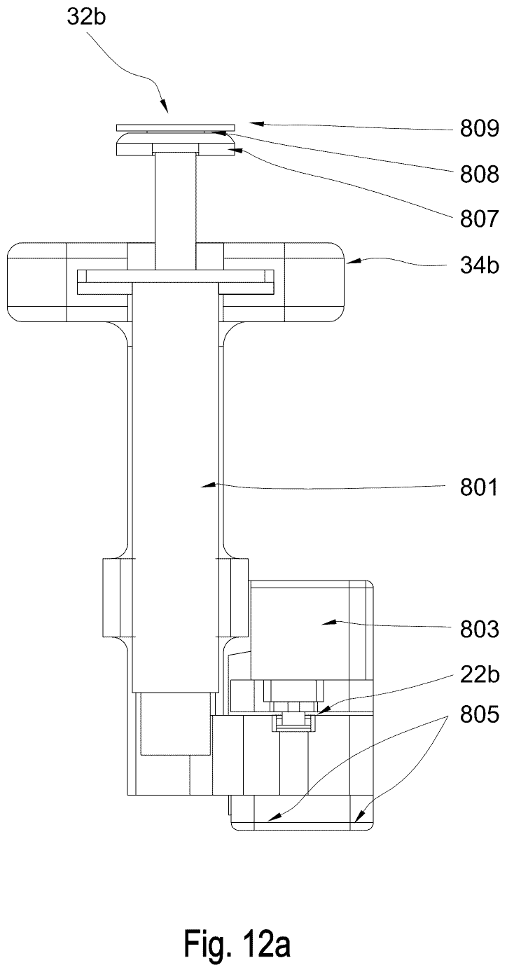

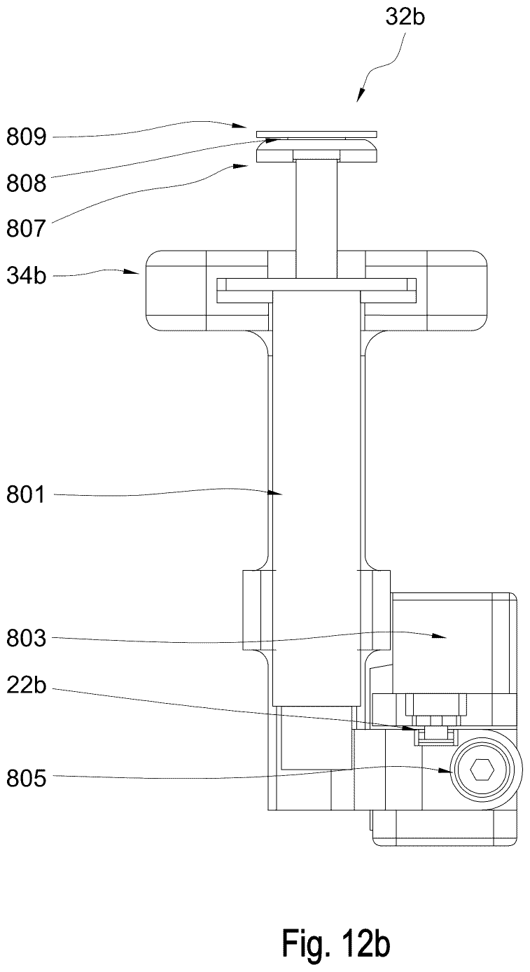

FIG. 12a shows a sectional view of a further tool variant, in particular a partial tool 20, having an end effector 32b in the context of a further embodiment of an ultrasonic robot 100 according to the disclosure and/or a further embodiment of a robot arm 10 according to the disclosure.

In particular, FIG. 12a shows a cost-effective disposable sensor system for measuring the needle penetration forces.

Injection syringe 32b, which has a needle 33b, is fixed to the matching syringe holder 34b. This is movably mounted with respect to attachment 803 of end effector sensor 22b, in particular configured as a force sensor.

In the embodiment according to FIG. 12a, a bearing with translational mobility along two guide pins 805 is implemented. As soon as the forces along the needle axis exceed the weight force of the syringe and syringe holder, the difference can be measured on end effector sensor 22b and/or calculated as a moment equilibrium about the axis of rotation using the measured values of end effector sensor 22b. If forces below the weight force of the syringe and the syringe holder are to be detected, it is preferable to pretension the syringe holder relative to end effector sensor 22b, for example using a spring-loaded pressure piece, wherein in particular the pretension should be above the expected maximum weight of the syringe and the syringe holder but well below the measurable maximum force of the force sensor. In addition, a further end effector sensor 808, for example a force measuring resistor, can be attached to the plunger of the syringe by means of a suitable connecting element 807. In particular, this measures the forces applied to plate 809 by user 102 and thus monitors the maximum pressure applied when injecting the medication. The main advantage of the concept shown, apart from its basic usability for various injection applications, is the low price of the components used: For example, a single-axis MEMS force sensor is already commercially available for around €30, suitable force measuring resistors for less than €10, and a microcontroller board for evaluating the sensor signals also for less than €10. This means that the syringe attachment with the integrated sensor system can be configured as a disposable component, which makes it easier to sterilize.

FIG. 12b shows a sectional view of a further tool variant, in particular a partial tool 20, having an end effector 32b in the context of a further embodiment of an ultrasonic robot 100 according to the disclosure and/or a further embodiment of a robot arm 10 according to the disclosure. The embodiment according to FIG. 12b corresponds to the embodiment of FIG. 12a, wherein in FIG. 12b a bearing with rotational mobility about a shoulder fitting screw 806 is implemented instead of the bearing with translational mobility along two guide pins 805 (FIG. 12a).

What is claimed is:

1. A robot arm, in particular a hands-on robot arm, comprising
a robot structure, wherein the robot structure comprises an arm link chain having at least one arm link and at least one joint, and wherein the robot structure further comprises a tool connected to the arm link chain for connection to at least one end effector;
at least one end effector sensor for detecting forces and/or torques acting on an end effector connected to the tool; and
at least one structure sensor for detecting forces and/or torques acting on the robot structure.

2. A robot arm according to claim 1, wherein the at least one end effector sensor and/or the at least one structure sensor is: a force sensor, a torque sensor or a force-torque sensor with two to six degrees of freedom.

3. The robot arm according to claim 1, wherein the arm link chain has a plurality of arm links with respective joints arranged therebetween.

4. The robot arm according to claim 1, further comprising a structure sensor between tool and arm link chain for detecting forces and/or torques acting on the tool.

5. The robot arm according to claim 1, wherein for at least one arm link, in particular for each arm link, a structure sensor for detecting forces and/or torques acting on the respective arm link is provided, preferably connected to the respective arm link.

6. The robot arm according to claim 1, further comprising at least one end effector detachably or non-detachably connected to the tool.

7. The robot arm according to claim 6, wherein an end effector sensor for detecting forces and/or torques acting on the tool is arranged between the at least one end effector, in particular between each end effector, and the tool.

8. The robot arm according to claim 1, further comprising a tool interface arranged between the arm link chain and the tool for connecting the tool to the tool interface, in particular in a detachable manner.

9. The robot arm according to claim 8, wherein a structure sensor for detecting forces and/or torques acting on the tool interface is arranged between the arm link chain and the tool interface.

10. The robot arm according to claim 1, further comprising a control unit for controlling the robot arm, wherein the control unit is configured for receiving and in particular processing the forces and/or torques detected by the end effector sensor and the structure sensor.

11. The robot arm according to claim 10, wherein the robot structure has at least one virtual fixture, in particular for restricting and/or damping the movement of the robot arm, the control unit being configured for adjusting the at least one virtual fixture on the basis of the forces and/or torques detected by the end effector sensor and by the structure sensor.

12. An ultrasonic robot comprising:
a robot arm according to claim 1,
an ultrasonic transducer connected to the tool as an end effector, and
preferably an injection needle connected to the tool as a further end effector.

13. A method for controlling a robot arm, in particular a robot arm according to claim 1 or an ultrasonic robot according to claim 12, comprising the steps of:
detecting forces and/or torques acting on an end effector of the robot arm,
detecting forces and/or torques acting on a robot structure of the robot arm,
determining, in particular calculating, interaction forces and/or interaction torques of the robot arm using the detected forces and/or torques.

14. The method according to claim 13, comprising the further step of:
outputting the interaction forces and/or interaction torques, in particular to an overall regulation of the robot arm.
